(19) United States
(12) Patent Application Publication
(10) Pub. No.: US 2018/0256287 A1
(43) Pub. Date: Sep. 13, 2018

(54) TOOL RFID RECOGNITION SYSTEM FOR DENTAL DEVICES

(71) Applicant: Kerr Corporation, Orange, CA (US)

(72) Inventors: Matteo R. Bosisio, Lugano (CH); Severin Reibenspiess, Turkenfeld (DE); Paolo Orsatti, Villa Luganese (CH); Filippo Piffaretti, Giubiasco (CH)

(21) Appl. No.: 15/721,793

(22) Filed: Sep. 30, 2017

Related U.S. Application Data
(60) Provisional application No. 62/470,441, filed on Mar. 13, 2017.

(51) Int. Cl. A61B 90/98; A61C 5/42

(52) U.S. Cl. CPC A61B 90/98; A61C 5/48; A61C 5/42

(57) ABSTRACT

A tool recognition system recognizes a tool selected for a surgical procedure. The tool includes a distal portion configured to perform at least part of the surgical procedure and a proximal portion including a first electronic module that stores tool-identifying electronic information. A surgical handpiece is configured to receive the tool and includes a second electronic module configured to communicate electronically with the first electronic module when tool is disposed in the handpiece. The first electronic module includes a coil that is configured as a radio-frequency identification (RFID) tag antenna. The second electronic module includes a coil that is configured as a radio-frequency identification (RFID) interrogator antenna. In one embodiment, the first electronic module is a transponder that includes a RFID chip and a RFID antenna. Another embodiment includes a surgical tool recognition system for a drill and the tool includes a file.

### CROSS REFERENCE TO RELATED APPLICATIONS

This application claims the benefit of U.S. Provisional Patent Application No. 62/470,441 filed Mar. 13, 2017, the entire contents of which are hereby incorporated by reference.

### FIELD OF TECHNOLOGY

Aspects of the disclosure relate generally to dental and/or surgical devices. More specifically, the disclosure relates to a system for tool recognition for tools used with dental and/or surgical devices.

Endodontic therapy, inclusive of root canal therapy, entails a series of treatments performed on a tooth. The treatments are generally performed on features within the tooth. The tooth may be compromised and/or infected. The treatments are directed to precluding onset of infection or removing infection. The treatments are directed to protecting the tooth from additional infection. Endodontic therapy may involve removal of nerve and pulp tissue from a root canal of the tooth. Endodontic therapy may involve cleaning, shaping, and decontamination of the tooth's root canal and its pulp chambers.

The shape of the formed root canal may be important in facilitating removal of dentinal debris and necrotic tissue. The shape may be important in facilitating irrigation cleaning processes. The shape may facilitate proper flow of gutta-percha (or similarly-purposed substances) and/or sealant during a subsequent obturation phase.

Objectives of root canal therapy may typically include:

a. creating a conical form of the root canal, from an access cavity (coronal) to an apical foramen;

b. preserving the natural curvature of the root canal;

c. avoiding foramen transportation; and/or

d. keeping the foramen as small as practical.

Generally, a means to achieve these objectives includes a rotary file attached to a contra-angle piece of an endodontic motor. (“Rotary” in this context may include reciprocation, with a file spinning and/or reciprocating, in rotary and/or linear fashion, and other motion types such as adaptive motion.) Typically, endodontic rotary devices include a console, a handpiece, a contra-angle head or piece, and a tool, such as a file (or series of files) to be used in a dental procedure. Other related therapies may require other tools, such as burrs and ultrasound tips, to be affixed to handpieces. Considerations that apply to files may apply as well to these and other endodontal, dental and/or surgical tools. The present embodiments may be applicable to such other endodontal, dental and/or surgical tools.

A large variety of types of rotary tools are available to a practitioner. The practitioner may use several types in a given procedure. Each type may require a specific set of motions to deliver its optimal performance. In addition, each tool type may be available in several sizes, such that the set of motions needed for a given tool type may require tuning as a function of specific tool size.

Several considerations may contribute to determination of the set of motions. Such considerations may include clinical considerations and other considerations. Clinical considerations may include root canal curvature and calcification. Clinical considerations may include specifics of a planned sequence of therapies to be applied to a given tooth or set of teeth. Other considerations may include device design considerations. For a file, device design considerations may include file cross-section, taper and flute. Device design considerations may include file helix angle, rake angle and pitch. Other considerations may include file material considerations. File material considerations may include file alloy and production-annealing. File material considerations may include file production surface hardening.

Files can withstand limited torsional stress. Beyond a limit of stress, a file may be prone to breakage. File breakage inside a root canal could cause major clinical issues. As a result, rotary devices are typically used at relatively low torque levels. The use of specialized files with differing shapes and sizes also helps avoid file breakage and improves root canal shaping. These files require a corresponding array of different motion settings to enable optimal performance and reliability.

Presently, endodontic motors on the market have several predefined type of motions. Dental health practitioners must manually select an appropriate setting, such as speed and adaptive motion or reciprocating motion, as a function of the type and size of rotary file they intend to use in a root canal procedure. Additionally, a maximum torque delivered by the motor must be manually selected to avoid potential file breakage in the root canal. Within the same procedure, different file sizes and types may be used in succession. The succession may involve many files. The succession may be rapid succession. The time lost in manually setting and resetting specific file motion parameters can be costly in both financial and medical terms. Such lost time may contribute to sub-optimal clinical practice. Suboptimal clinical practice may also result from a practitioner inserting into the handpiece a file of a type incompatible with the handpiece and/or with a desired surgical procedure. Incompatibility with the handpiece may include geometric incompatibility. Incompatibility with the handpiece may include incompatibility with operation parameters of the handpiece, including motion parameters.

If an incorrect motion parameter is set, the file may break inside the tooth. A portion of the file left inside the tooth can create a blockage preventing a complete canal cleaning which can in turn lead to further complications. Human error and avoidance of inconvenience are two additional issues associated with manual selection (calibration) of motion settings to file type.

Endodontal, dental and/or surgical tools may typically be manufactured well before their use in a clinical setting. The tools may require one or more sterilization procedures prior to clinical use. Significant time may pass prior to clinical use, potentially increasing risk of compromised tool sterility and/or structural integrity. It may therefore be desirable to keep track of time elapsed since manufacture and/or sterilization, as well as the number of sterilization cycles a tool has been subjected to.

It may be necessary to track location of a tool within a manufacturing facility, within a distribution facility such as a warehouse, and/or within a practitioner's facility. At the practitioner's facility, tracking of age, usage and sterilization cycle count may be necessary and/or required for best practice and/or by regulations. At the practitioner's facility, identification of tool-type and tool-history may be desirable at a surgical deck from which tools are selected to be used with the handpiece for the desired surgical procedure.

It would be desirable, therefore, to provide apparatus and methods for a reliable and automatic detection of endodontal tool properties, including identity (tool type), location, prior history and age. It would further be desirable, therefore, to provide apparatus and methods for a reliable and automatic setting of motion parameters based on the type, inclusive of size, of a tool inserted into an orthodontal, dental or surgical handpiece.

### SUMMARY OF THE DISCLOSURE

It is an object to provide apparatus and methods for automatically calibrating or setting parameters of an orthodontal, dental or surgical handpiece to the unique settings useful for a tool removably coupled to the handpiece. The calibration may be effected by receiving information stored in the tool. The information may be stored electronically. The information may be received electronically from the tool. The information may be updated electronically.

It is also an object to provide apparatus and methods for automatically recognizing a orthodontal, dental or surgical tool. Recognizing the tool may include tracking the tool in settings of storage (for example, in a warehouse, in a practitioner's facility), tool- preparation (for example, in an equipment sterilizer), and/or clinical use (for example, in the surgical deck). Tracking the tool may be effected by receiving information electronically stored in the tool. The information may include identification of the tool. The information may include status of the tool. Status of the tool may include history of tool-usage. Status of the tool may include a record of tool-preparation (for example, a number of thermal sterilization cycles undergone by the tool). The information may be received electronically and updated electronically.

### BRIEF DESCRIPTION OF THE DRAWINGS

The objects and advantages will be apparent upon consideration of the following detailed description, taken in conjunction with the accompanying drawings, in which:

FIG. 1 is a schematic representation of a dental rotary device;

FIG. 2 is a partial cross-sectional view of a handpiece receiving a tool, with some features shown in perspective;

FIG. 3A is a partial cross-sectional view of a transponder;

FIG. 3B is a top plan view of another transponder;

FIG. 4 is a perspective lateral view of an extended file, with some internal features represented in phantom;

FIG. 5 is a perspective lateral view of an extended file, with a cutaway showing some internal features and with some internal features represented in phantom;

FIG. 6 is a perspective lateral view of a file, with some internal features represented in phantom;

FIG. 7 is a perspective lateral view of a file, with some internal features represented in phantom;

FIG. 8 is a perspective lateral view of a file, with some internal features represented in phantom;

FIG. 9 is an exploded perspective lateral view of a disassembled multi-part extended file, with some internal features represented in phantom, and also an assembled view of the file with indication of internal features and insert;

FIG. 10 is a partial cross-sectional view of a handpiece receiving a tool, with some features shown in perspective;

FIG. 11 is a partial cross-sectional view of apparatus, with some features shown in perspective;

FIG. 12 is a partial cross-sectional view of a handpiece, with some features shown in perspective, and a detail of part of the handpiece shown in inset, with a cutaway providing a view of some internal features;

FIG. 13 is a perspective view of a handpiece and tools on surgical deck, with some internal features represented and with several locations of tools, transponders, reading antennae and associated electromagnetic fields indicated; and

FIG. 14 is a block diagram of a dental tool recognition system.

### DETAILED DESCRIPTION OF THE DISCLOSURE

Apparatus and methods for dental procedures are provided. The apparatus may be used to perform one or more steps of the methods. The methods may include methods for manufacture of one or more of the apparatus.

Exemplary embodiments are shown and described below. Features, including structures, materials, volumes, functions and other attributes that are shown and described in connection with any of the embodiments may be combined, in whole or in part, with each other or included, in whole or in part, in other embodiments.

Apparatus and methods described herein are illustrative. Some embodiments may omit steps shown and/or described in connection with the illustrative methods. Some embodiments may include steps that are neither shown nor described in connection with the illustrative methods. Illustrative method steps may be combined. For example, one illustrative method may include steps shown in connection with another illustrative method.

Some apparatus may omit features shown and/or described in connection with illustrative apparatus. Some embodiments may include features that are neither shown nor described in connection with the illustrative methods.

Features of illustrative apparatus may be combined. For example, one illustrative embodiment may include features shown in connection with another illustrative embodiment.

An apparatus may involve some or all of the features of any of the illustrative apparatus and/or some or all of the steps of any of the illustrative methods. A method may involve some or all of the features of any of the illustrative methods and/or some or all of the steps of any of the illustrative apparatus.

The apparatus may include, and the methods may involve, a surgical tool recognition system. The surgical tool recognition system may include a surgical tool. The surgical tool may be selected for a particular surgical procedure. The surgical tool may define a tool longitudinal axis. The surgical tool may include a distal portion. The distal portion may be configured to perform at least part of the surgical procedure.

The surgical tool may be a dental tool. The surgical tool may be an endodontal tool. The surgical tool may be a rotary tool. The surgical tool may be selected for the procedure from a wide variety of files, broaches, burrs, and other suitable tools. Other suitable tools may include reamers and drill bits. Other suitable tools may include dental curing lights. Other suitable tools may include lasers. Other suitable tools may include ultrasonic tips. Other suitable tools may include vibratory cutters.

The surgical tool may include a proximal portion. The proximal portion may include a first electronic module. The first electronic module may be disposed within the tool, and may be disposed within the proximal portion. The first electronic module may be disposed entirely within the tool, and in one embodiment, entirely within the proximal portion.

The first electronic module may include a first module coil. The first module coil may define a first module coil axis. The first module coil axis may be substantially collinear with the tool longitudinal axis. The first module coil may include a radio-frequency identification (RFID) tag antenna. The first module coil may be configured as the radio-frequency identification (RFID) tag antenna.

The first electronic module may store tool-characterizing electronic information. The first electronic module may store tool-identifying electronic information.

The system may include a surgical handpiece. The handpiece may be configured to receive the tool. The tool may be received in the handpiece. The handpiece may be configured to drive the tool during the procedure.

The handpiece may be configured to control a function of the distal portion of the tool during the procedure. The function may include rotary speed. The function may include torque. The function may include acceleration rate. The function may include deceleration rate. The function may include reciprocation linear stroke length. The function may include clockwise rotary reciprocation angular extent. The function may include counterclockwise rotary reciprocation angular extent. The function may include any other suitable surgical tool functions. Other suitable surgical tool functions may include rotary and/or linear reciprocation frequency. Other suitable surgical tool functions may include light emission wavelength. Other suitable surgical tool functions may include light emission intensity. Other suitable surgical tool functions may include mechanical vibration frequency.

The handpiece may include a second electronic module. The second electronic module may include a second module coil that defines a second module axis.

The second module coil may include a radio-frequency identification (RFID) interrogator antenna. The second module coil may be configured as the radio-frequency identification (RFID) interrogator antenna. The second electronic module may be configured to communicate electronically with the first electronic module.

The second module coil may be disposed within the handpiece. The second module coil may be disposed entirely within the handpiece. The handpiece may be configured to contain the second electronic module. The handpiece may be configured to entirely contain the second electronic module. A region about the second module axis may be configured to receive the proximal portion of the tool.

When the handpiece receives the tool, some or all of the first electronic module may be disposed within the second module coil. Some or all of the first module coil may be disposed within the second module coil.

The tool may be removably coupled to the handpiece. The tool may be disposed in the handpiece. With the tool disposed in the handpiece, the tool longitudinal axis may be disposed substantially collinear with the second module axis. The first module coil axis may be substantially collinear with the second module axis.

The second electronic module may activate the first module. The second electronic module may power the first electronic module. The second electronic module may activate the first module to electronically transmit the tool-identifying information. The first module may transmit a version of the tool-identifying information. The first electronic module may transmit the version of the tool-identifying information to the second electronic module. The second electronic module may receive the version of the tool-identifying information. The version of the tool-identifying information may correspond to some or to all of the tool-identifying information.

The handpiece may be configured to control the function of the distal portion of the received tool on a basis of the version of the tool-identifying information transmitted by the first electronic module, such as a transponder.

The surgical tool recognition system may include a data processing module. The data processing module may store sets of operating parameter values of the handpiece appropriate to one or more specific surgical tools. The data processing module may be in communication with the second electronic module. The data processing module may be configured to retrieve a value appropriate to the selected surgical tool. The data processing module may be configured to retrieve, on the basis of the information transmitted, the value appropriate to the selected surgical tool. The value of the operating parameter of the handpiece may be set to correspond to the tool-identifying information transmitted by the first electronic module.

The first electronic module may store non-tool-identifying electronic information. The non-tool-identifying information may include a record of use of the tool. The non-tool-identifying information may include any other suitable data. Any other suitable data may include data corresponding to an environmental condition of the tool.

With the tool disposed in the handpiece, the first electronic module may electronically transmit the non-tool-identifying information. The first electronic module may transmit a version of the non-tool-identifying information to the second electronic module. The second electronic module may receive the version of the non-tool-identifying information. The non-tool-identifying information may correspond to some or to all of the non-tool-identifying information.

The handpiece may be configured to control the function of the distal portion of the received tool on a basis of the version of the non-tool-identifying information transmitted by the first electronic module. A value of the operating parameter of the handpiece may be set to correspond to the non-tool-identifying information.

The apparatus may include, and the methods may involve, an endodontic tool recognition system. The system may include an endodontic tool. The tool may define a longitudinal axis. The tool may include a distal portion. The distal portion may be configured to perform at least part of an endodontic procedure. The tool may include a shank portion. The shank portion may include a first electronic module. The first electronic module may store tool-characterizing electronic information. The tool-characterizing electronic information may include tool-identifying information. One or more than one portions of the first electronic module may be substantially collinear with the longitudinal axis.

The system may include an endodontic handpiece. The handpiece may be configured to receive the shank. The tool may be received in the handpiece. The handpiece may be configured to control a function of the received tool. The handpiece may be configured to control a function of the received tool during the procedure. The handpiece may include a second electronic module. The second electronic module may be configured to communicate electronically. The second electronic module may be configured to communicate electronically with the first electronic module.

With the shank disposed in the handpiece, the longitudinal axis may be substantially collinear with an axis of the second electronic module. With the shank disposed in the handpiece, the first electronic module may transmit electronically a version of the information. With the shank disposed in the handpiece, the first electronic module may transmit electronically a version of the information to the second electronic module. The version may determine a value of an operating parameter of the handpiece. The value may correspond to the function.

The apparatus may include, and the methods may involve, an endodontic file recognition device. The device may include an endodontic file. The file may define a longitudinal axis. The file may include a portion. The portion may be fluted. The portion may be configured to perform at least part of a root canal procedure.

The file may include a shank portion. The shank portion may fully or partly encase a radio-frequency identification (RFID) tag. The device may include an RFID tag antenna coil. The file may include the RFID tag antenna coil. The shank portion may include the RFID tag antenna coil. The shank portion may fully or partly encase the RFID tag antenna coil. The RFID tag antenna coil may be disposed substantially collinear with the longitudinal axis.

The device may include an endodontic drill. The drill may be configured to receive the shank. The drill may be configured to removably receive the shank. The drill may be configured to control at least one function of the received file during the procedure. The drill may include an RFID interrogator antenna coil. The RFID interrogator antenna coil may be configured to communicate electronically with the RFID tag.

With the shank received in the drill, the interrogator coil may be substantially collinear with the longitudinal axis. With the shank received in the drill, the RFID tag may transmit electronically a version of information identifying the file to the interrogator coil. The version may determine one or more than one operating parameters of the drill. The operating parameter may correspond to the at least one function.

The apparatus may include, and the methods may involve, an endodontic drill. The drill may include a first section. The first section may be configured to receive a shank of an endodontic file. The first section may be configured to removably receive a shank of an endodontic file. The file may fully or partly encase a radio-frequency identification (RFID) tag. The first section may be configured to drive motion of the file during a root canal procedure.

The drill may include a second section. The second section may cover the first section. The second section may include an RFID interrogator antenna coil. The RFID interrogator antenna coil may be configured to accommodate a girth of the shank. The RFID interrogator antenna coil may be configured to communicate electronically with the RFID tag.

With the shank received in the first section, the RFID tag may be disposed at least partly within the interrogator coil. With the shank received in the first section, the RFID tag may transmit electronically a version of information identifying the file. With the shank received in the first section, the RFID tag may transmit electronically a version of information identifying the file to the interrogator coil. The version may determine an operating parameter of the drill. The operating parameter may correspond to the motion.

The apparatus may include, and the methods may involve, an endodontic file for use in a root canal procedure. The file may include a portion. The portion may be fluted. The portion may be configured to perform at least part of the root canal procedure.

The file may include a shank portion. The shank portion may be configured to couple with an endodontic drill. The shank portion may be configured to releasably couple with an endodontic drill. The shank portion may at least partly encase a radio-frequency identification (RFID) tag. The shank portion may at least partly encase an RFID tag antenna coil. The tag may store electronic information. The electronic information may identify the file. The drill may be configured to control a motion of the file. The drill may include an RFID interrogator antenna coil. The RFID interrogator antenna coil may be configured to accommodate a girth of the shank portion. The RFID interrogator antenna coil may communicate electronically with the RFID tag.

With the shank portion coupled to the drill, the tag coil may be disposed at least partly within the interrogator coil.

With the shank portion coupled to the drill, the interrogator coil may electronically activate the RFID tag.

With the shank portion coupled to the drill, the interrogator coil may receive electronically a version of the information from the RFID tag. The version may determine a characteristic of the motion of the file.

The characteristic of the motion may be selected from an electronic memory. The characteristic of the motion may be selected from an electronic memory to correspond to the version of the information received from the tag.

The characteristic of the motion may be optimized for the file. The characteristic of the motion may be optimized for the file as identified by the version of the information received from the RFID tag.

The characteristic of the motion may include rotary speed. The characteristic of the motion may include torque. The characteristic of the motion may include acceleration rate. The characteristic of the motion may include deceleration rate. The characteristic of the motion may include reciprocation linear stroke length. The characteristic of the motion may include clockwise reciprocation angular extent. The characteristic of the motion may include counterclockwise reciprocation angular extent. The characteristic of the motion may include reciprocation frequency.

The apparatus may include, and the methods may involve, a surgical tool for use in a surgical procedure. The tool may define a tool longitudinal axis. The tool may include a distal portion. The distal portion may be configured to perform at least part of the procedure.

The tool may be a dental tool. The tool may be an endodontal tool. The tool may be selected for the procedure from a variety of files, broaches, burrs, reamers and drill bits.

The tool may include a proximal portion. The proximal portion may be configured to couple with a surgical handpiece. The proximal portion may be configured to releasably couple with the surgical handpiece. The proximal portion may include a radio-frequency identification (RFID) tag. The tag may store electronic information characterizing the tool. The proximal portion may include an RFID tag antenna.

The RFID tag may be disposed within a transponder casing. The RFID tag antenna may be disposed within the transponder casing. The transponder casing may enclose a capacitor. The capacitor may be in electronic communication with the RFID tag. The capacitor may be in electronic communication with the RFID tag antenna.

An axis of the RFID tag antenna may be substantially collinear with the longitudinal tool axis. The RFID tag and RFID tag antenna may be disposed relative to each other substantially linearly along the longitudinal tool axis. The RFID tag and RFID tag antenna may be disposed non-coterminously relative to each other.

The RFID tag and RFID tag antenna may be disposed relative to each other in a coil-and-chip configuration.

The RFID tag and RFID tag antenna may be used for high radio frequency applications. The RFID tag and RFID tag antenna may be used for low frequency radio applications. The transponder may be configured as a near-field communication device. The near-field communication transponder may communicate with the RFID interrogator antenna coil in proximity to the RFID interrogator antenna coil. The near-field communication transponder may communicate with the RFID interrogator antenna coil given axial alignment of the transponder and interrogator. The transponder may be configured as a far-field communication device. The far-field communication transponder may communicate with the RFID interrogator antenna coil at greater distances and/or with less axial alignment than the near-field communication transponder.

The handpiece may be configured to control a motion of the tool during the procedure. The handpiece may include an RFID interrogator antenna coil. The RFID interrogator antenna coil may be configured to accommodate a girth of the distal portion of the tool. The RFID interrogator antenna coil may be configured to electronically communicate with the RFID tag.

When the proximal portion of the tool is coupled with the handpiece, the tag antenna may be disposed at least partly within the interrogator coil. The RFID tag may be activated via the interrogator coil. The RFID tag may be activated inductively via the interrogator coil. The interrogator coil may electronically receive a version of the information from the RFID tag. The version may determine a characteristic of the motion of the tool during the procedure.

The characteristic of the motion may be selected from an electronic memory to correspond to the version of the information received from the RFID tag. The characteristic of the motion may be optimized for the tool as identified by the version of the information received from the RFID tag.

The characteristic of the motion may be rotary velocity. The characteristic of the motion may be torque. The characteristic of the motion may be acceleration rate. The characteristic of the motion may be deceleration rate. The characteristic of the motion may be reciprocation linear stroke length. The characteristic of the motion may be clockwise reciprocation angular extent. The characteristic of the motion may be counterclockwise reciprocation angular extent. The characteristic of the motion may be reciprocation frequency.

The apparatus may include, and the methods may involve, a method for setting an operating parameter of an endodontic handpiece. The handpiece may be an endodontic drill.

The method may include selecting an endodontic tool for performing an endodontic procedure. The tool may include an end portion. The end portion may be configured to contact a tooth during at least part of the endodontic procedure. The end portion may be configured to perform at least part of the endodontic procedure. The end portion may be fluted.

The tool may include a shank portion. The shank portion may be configured to couple with the endodontic handpiece. The shank portion may be configured to removably couple with the endodontic handpiece. The shank portion may include a radio-frequency identification (RFID) tag. The shank portion may fully or partly contain the RFID tag. The tag may store electronic information. The information may be tool-characterizing information. The information may identify the tool.

The method may include coupling the tool with the handpiece. The method may include removably coupling the tool with the handpiece. The handpiece may be configured to control a motion of the tool during the procedure. The handpiece may include an RFID interrogator antenna coil. The interrogator antenna coil may be configured to accommodate a girth of the shank portion. The interrogator antenna coil may be configured to communicate electronically with the RFID tag. The coupling may dispose at least part of the RFID tag within the interrogator antenna coil. The RFID tag may be associated with a tag antenna coil. The RFID tag may be in electronic communication with the tag antenna coil. The shank portion may fully or partly contain the tag antenna coil. The coupling may dispose at least part of the tag antenna coil within the interrogator antenna coil.

The tool may define a longitudinal axis. The tag antenna coil may be substantially collinear with the longitudinal axis. The coupling may dispose the longitudinal axis substantially parallel to a coil axis of the interrogator antenna coil.

The method may include electronically activating the tag. The activating may be accomplished via the interrogator antenna coil. The activating may result in the RFID tag transmitting the electronic information. The transmitting may be accomplished via the tag antenna coil.

The method may include electronically receiving the transmitted version. The receiving may be accomplished by the RFID interrogator antenna coil.

The method may include retrieving from electronic memory, on the basis of the received version of characterizing information, a value of the operating parameter corresponding to the motion of the selected tool during the procedure.

The method may include electronically setting the operating parameter to conform to the retrieved value.

The shank portion may include a first material. The first material may include a metallic alloy. The first material may include a high-stress-tolerant metal. The first material may include a non-metallic substance. The first material may include a polymer. The first material may include a ceramic.

The RFID tag and the tag antenna coil may be encased within the shank portion in a second material. The RFID tag and the tag antenna coil may be components of a transponder. The second material may encase the transponder. The second material may include a glass.

The end portion may include a metallic alloy. The shank portion may include a material different from the material of the end portion. The end portion may be fixed in mechanical association with the shank portion.

The shank portion may include a material that facilitates electronically transmitting the version of tool-characterizing information. The material may facilitate electronically receiving the transmitted version.

The shank portion may be geometrically configured to facilitate electronically transmitting the version to the second electronic module. The shank portion may be geometrically configured to facilitate the second electronic module electronically receiving the version. The shank may feature one or more than one windows. The window(s) may be open window(s). The window(s) may lie along a circumference of the shank. The window(s) may be disposed between the tag antenna coil and the interrogator antenna coil. The windows may facilitate electronic communication between the transponder and the interrogator antenna coil.

The apparatus may include, and the methods may involve, a surgical tool recognition system. The system may include a surgical tool. The tool may be selected for a surgical procedure. The tool may include a dental tool. The dental tool may include a file. The dental tool may include a broach. The tool may include a dental burr. The tool may include a reamer. The tool may include a drill bit. The tool may include any suitable surgical tool. Any suitable surgical tool may include a vibratory cutter.

The tool may include a distal portion. The distal portion may be configured to perform at least part of the surgical procedure.

The tool may include a proximal portion. The proximal portion may include a first electronic module. The first electronic module may store tool-associated electronic information. The first electronic module may be disposed within the tool. The first electronic module may be disposed entirely within the tool. The first electronic module may include a radio-frequency identification (RFID) tag antenna.

The system may include a second electronic module. The second electronic module may include an RFID interrogator antenna. The second electronic module may be configured to communicate electronically with the first electronic module.

The tool may be disposed in proximity to the second electronic module. The proximity of the first electronic module to the second electronic module may facilitate electronic communication between the first electronic module and the second electronic module. With the tool disposed in proximity to the second electronic module, the first electronic module may transmit electronically a version of the information. The version of information may be received by the second electronic module. A system parameter may be electronically set corresponding to the version of information.

The second electronic module may be associated with a surgical handpiece. The handpiece may be configured to control a function of the tool. The handpiece may be configured to control a function of the tool during the surgical procedure.

The second electronic module may be disposed at least partly internal to the surgical handpiece. The system parameter may correspond to the function of the tool.

The second electronic module may be disposed entirely within the handpiece. The handpiece may be configured to contain the second electronic module. The handpiece may be configured to entirely contain the second electronic module. The second electronic module may be configured to receive the proximal portion of the tool.

The second electronic module may be disposed at least partly external to the surgical handpiece. The third module may be disposed at least partly external to the surgical handpiece, in addition to an RFID interrogator module disposed internal to the handpiece. The second electronic module may be disposed on a surface of the handpiece opposite a position of the RFID interrogator module disposed internal to the handpiece. The second electronic module may be disposed on an external surface of the handpiece. The second electronic module may be disposed on a posterior surface of the handpiece away from tool-receiving sections of the handpiece, such as on part of a hand-grip area. The second electronic module may be configured to facilitate a proximity with the tool, with the tool being external to the handpiece. The proximity may include a distance of less than about 1 meter. The proximity may include a distance of about 15 cm.

The system parameter may correspond to a confirmation of a status of the tool. The status of the tool may include the identity of the tool. The status of the tool may include a measure of prior usage of the tool. The status of the tool may include a measure of readiness of the tool for use in the surgical procedure.

The second electronic module may be disposed in a distribution facility. The facility may be configured to store the tool prior to distribution. The facility may be configured to store the tool prior to distribution to a practitioner of the surgical procedure. The system parameter may correspond to a location of the tool within the distribution facility. The system parameter may correspond to an inventorying of the tool within the distribution facility.

The second electronic module may be disposed in association with an equipment sterilizer. The second electronic module may be disposed in the sterilizer. The sterilizer may be configured to sterilize the tool prior to packaging of the tool. The sterilizer may be configured to sterilize the tool prior to use of the tool in the surgical procedure. The system parameter may correspond to a location of the tool within the equipment sterilizer. The system parameter may correspond to a count of sterilization cycles to which the tool has been subjected. The system parameter may correspond to completeness of a sterilization cycle. The system parameter may correspond to completeness of a sterilization cycle prior to the surgical procedure.

The second electronic module may be disposed in a thermal disinfector. The disinfector may be configured to disinfect the tool prior to use of the tool in the surgical procedure.

The second electronic module may be disposed in a dental treatment unit. The treatment unit may be configured for implementation of the surgical procedure.

The second electronic module may be disposed in a storage holder. The second electronic module may be disposed in a storage holder in a practitioner's facility. The holder may be configured to hold the tool. The holder may be configured to hold the tool prior to use of the tool in the surgical procedure. The holder may be configured as a surgical deck from which the tool may be selected from other similar and/or different tools to be used with the handpiece for the surgical procedure. The system parameter may correspond to a confirmation of a status of the tool. The status of the tool may include the identity of the tool. The status of the tool may include a location of the tool. The status of the tool may include a location of the tool within the practitioner's facility. The system parameter may correspond to an inventorying of the tool within the practitioner's facility. The status of the tool may include a measure of prior usage of the tool. The status of the tool may include a measure of readiness of the tool for use in the surgical procedure.

The second electronic module may activate the first electronic module to transmit information. The information may include data corresponding to an environmental condition of the tool. With the tool disposed in proximity to the second electronic module, the information may be electronically modified. The information may be modified via a transmission from the second electronic module.

Apparatus and methods will now be described in connection with the FIGS., which form a part hereof. The FIGS. show illustrative features of apparatus and/or methods. The features are illustrated in the context of selected embodiments. It will be understood that features shown in connection with one of the embodiments may be practiced along with features shown in connection with another of the embodiments.

Apparatus and methods described herein are illustrative. Apparatus and methods may involve some or all of the features of the illustrative apparatus and/or some or all of the steps of the illustrative methods. The steps of the methods may be performed in an order other than the order shown and described herein. Some embodiments may omit steps shown and described in connection with the illustrative methods. Some embodiments may include steps that are not shown and/or not described in connection with the illustrative methods. Illustrative method steps may be combined. For example, one illustrative method may include steps shown in connection with another illustrative method.

The apparatus and methods will be described in connection with embodiments and features of illustrative devices. It is to be understood that other embodiments may be utilized and that structural, functional and procedural modifications may be made.

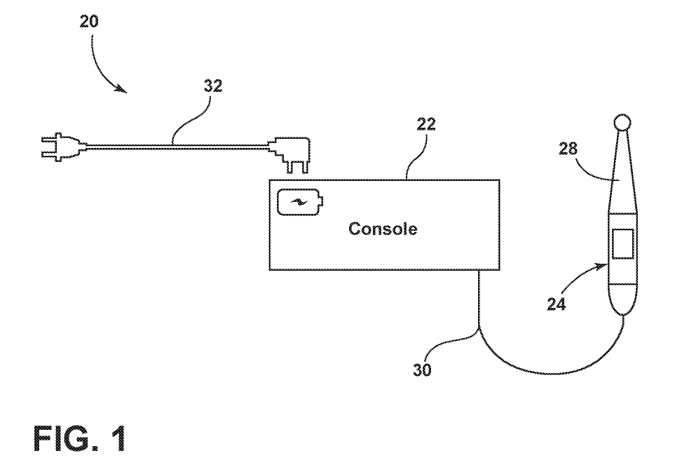

FIG. 1 depicts a dental rotary device 20 comprising a console 22 and a handpiece 24 including a contra-angle portion 28. The handpiece 24 is typically connected at one end to the console 22 by a cable 30, which is in turn wired to a power outlet by a power connector 32. Alternatively and/or additionally, the handpiece 24 may communicate wirelessly with console 22. The handpiece 24 may be battery-powered in place of the cable 30. The handpiece 24 may be connected at its other end to a replaceable tool, such as a spinning file or, over a course of a dental procedure such as endodontic therapy or a prophylaxis, a series of tools including files. In some embodiments, the dental rotary device 20 is a contra-angle device.

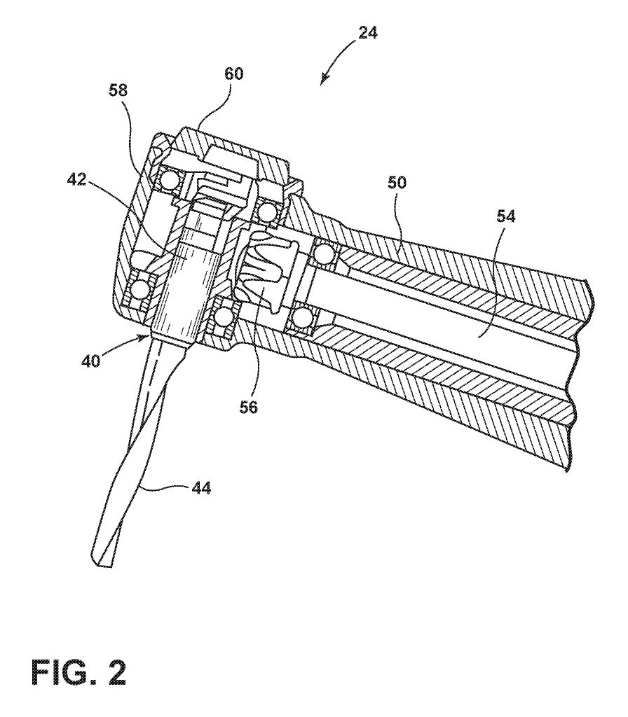

FIG. 2 shows a tool 40, with a shank 44 and flutes 46, disposed in a handpiece 24. The handpiece 24 includes a head sleeve 50, an intermediate drive shaft 54, a drive gear 56, a contra-angle cover 58, and a button 60. The configuration shown may be typical of equipment currently in use. Handpiece operating settings appropriate for a particular tool, featuring a file and file flutes, may have to be set manually.

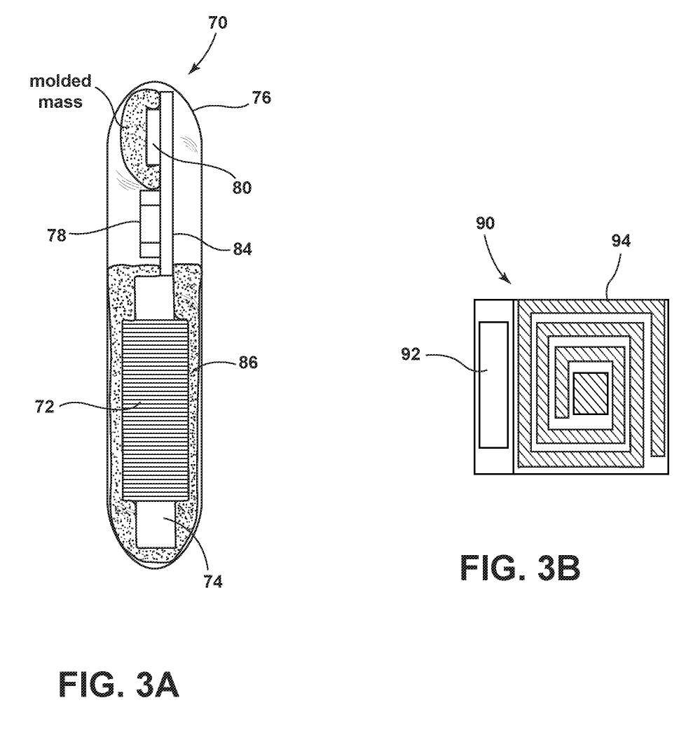

FIG. 3A shows a transponder 70. The transponder 70 features a tag antenna coil 72 wrapped about a ferrite rod 74. The tag antenna coil 72 is depicted as non-coterminus with an RFID tag. The transponder 70 shown is glass-encased with a glass housing 76 and includes a chip capacitor 78 and an RFID tag chip 80 in electronic communication through a printed circuit board 84 with the tag antenna coil 72. The tag antenna coil 72 may inductively receive power from an antenna (not shown) external to the transponder 70. The capacitor 78 may be charged from the tag antenna coil 72. The RFID tag chip 80 may be powered by the capacitor 78. The RFID tag chip 80 may contain tool-characterizing electronic information. The information, or a version thereof, may be transmitted by the powered RFID tag chip 80 via the tag antenna coil 72. Electronic components of the transponder 70 may be protected and/or cushioned (both in manufacture and in use) internal to the glass housing 76 by a molded mass of hard material and/or by a soft adhesive fill 86. The soft adhesive fill 86 may occupy a volume within the glass that is similar to, or different from, the volume depicted, covering components of the transponder in a fashion similar to, or different from, that depicted.

FIG. 3B shows a top view of a transponder 90 with a coil-and-chip arrangement. The transponder 90 includes a RFID chip 92 and an RFID antenna 94. Such a transponder 90 possesses a smaller profile, with smaller dimensions, than the configuration of transponder 70 shown in FIG. 3A. Embedment in a tool, such as file of a transponder 90 of the type and/or size depicted in FIG. 3B or of small (particularly, longitudinally short) versions of the type depicted in FIG. 3A, may minimize extension of file shank, relative to overall file length, to be accommodated in the contra-angle portion (not shown) of a handpiece. Such embedment allows for a correspondingly smaller second electronic module to be accommodated in the contra-angle portion 28 of the handpiece 24.

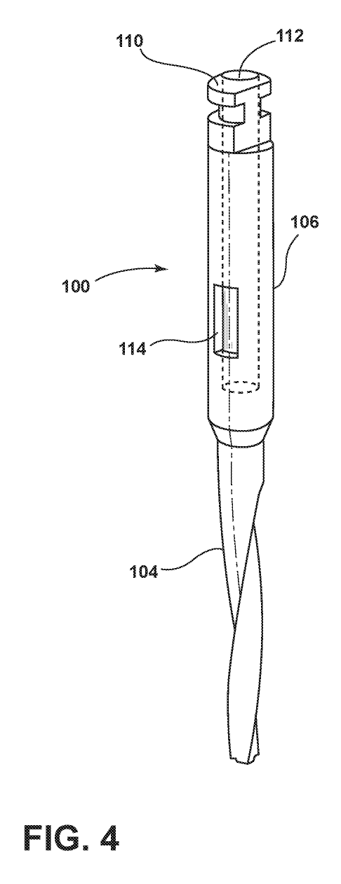

FIG. 4 shows an extended file 100, with flutes 104 and an extended file shank 106. At its proximal end 110, the extended file shank 106 includes an opening to an access bore 112. The access bore is configured to house the transponder 70 shown in FIG. 3A. The extended file shank 106 includes an open window 114. The extended file shank may be longer than the shank 42 depicted in FIG. 2. Extension of the extended file shank 106 may facilitate accommodating an electronic module, such as the transponder 70 depicted in FIG. 3A.

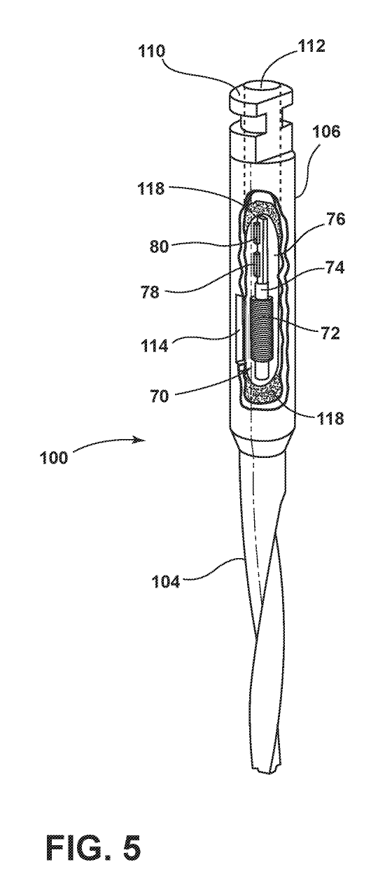

FIG. 5 shows the extended file 100 of FIG. 4 with the transponder 70 of FIG. 3A disposed within the access bore 112. The transponder 70 is inserted through the top (proximal) end opening of the access bore 112. The extended file 100 includes an extended file shank 106 and file flutes 1-4. Extension of the file shank 106 over standard file shanks is necessitated by the length of the transponder 70. Smaller transponders, such as the transponder 90 shown in FIG. 3B, or smaller versions of the transponder 70 shown in FIG. 3A, may allow for diminished extension of the file shank 106 and for use of such transponders in tools not readily produced in extended configuration. The access bore 112 includes a bore fill or cushion 118 external to the glass housing 76 of the transponder 70. The fill/cushion 118 supports and protects the transponder 70 disposed within the extended file 100. The transponder 70 includes the RFID tag chip 80, the chip capacitor 78, and the ferrite rod 74. The tag antenna coil 72 is exposed through the open window 114. The open window 114 facilitates electronic communication to and from the tag antenna coil 72.

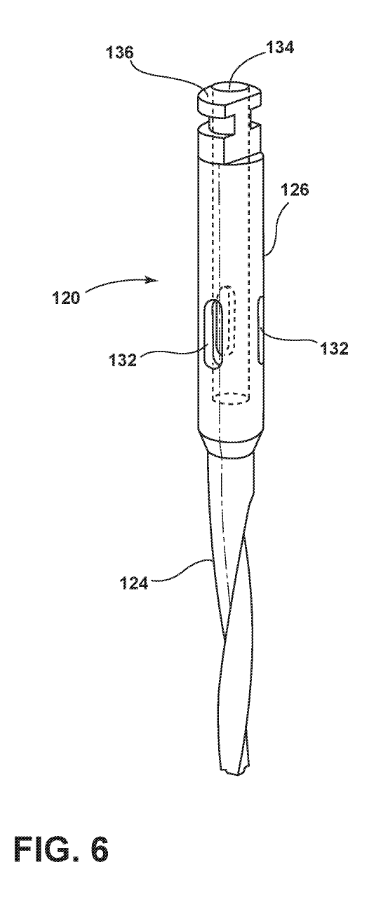

FIGS. 6-9 show a variety of configurations of extended files that facilitate electronic communication to and from the tag antenna coil 72.

FIG. 6 shows an extended file 120, with flutes 124 and an extended file shank 126. The extended file shank 126 features several open windows 132 and an access bore 134 open from the top (proximal) end 136 of the file. The several open windows 132 may provide a total unobstructed area facilitating electronic transmission to and from a transponder (not shown) disposed in the file.

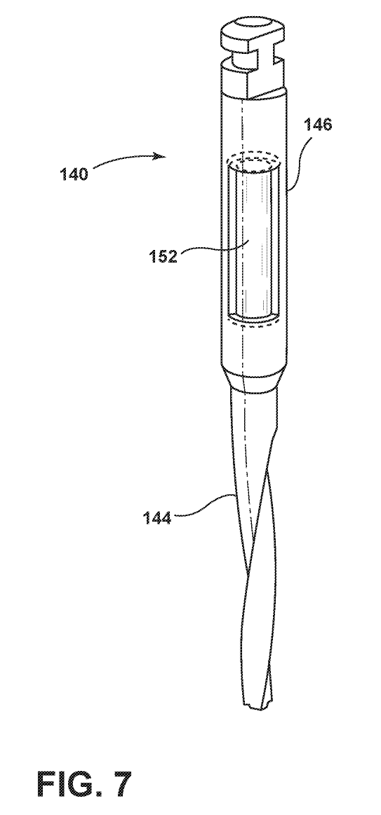

FIG. 7 shows an extended file 140, with flutes 144 and an extended file shank 146. The extended file shank 146 features a transverse access open window 152 configured for insertion of a transponder 70 (example shown in FIG. 3A) in a direction transverse to a longitudinal axis of the file 140. The large open window 152 provides a substantial area of unobstructed electronic transmission to and from a transponder (not shown) disposed in the file 140.

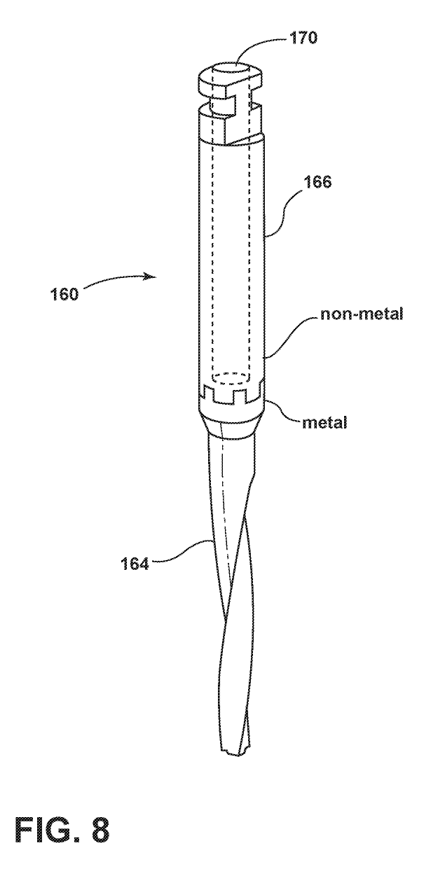

FIG. 8 shows a multi-part extended file 160, with flutes 164 and an extended file shank 166 defining a top-end opened access bore 170. The file 160 features a non-metallic extended file shank 166. The non-metallic file shank 166 may include a ceramic or polymer material that facilitates electronic transmission to and from a transponder (not shown) disposed in the file 160. The file 160 features metallic flutes 164. The metallic file flutes 164 and the non-metallic file shank 166 are shown fixedly mechanically associated in FIG. 8.

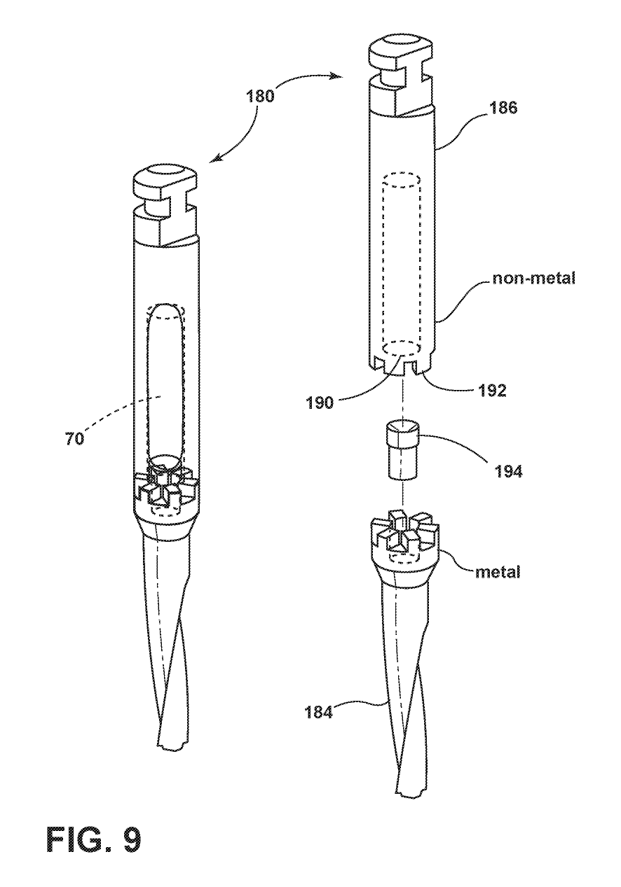

FIG. 9 shows assembled and (exploded) disassembled views of a multi-part extended file 180, with flutes 184 and an extended file shank 186. The extended file 180 features a non-metallic extended file shank 186. The file 180 features metallic flutes 184. The file 180 features a bottom-accessed access bore 190 that opens from a distal end 192 of the file shank 186. A transponder (indicated in outline in the assembled view) may be inserted into the file shank 186 through a bottom opening of the access bore 190, and may be supported from below by a joining member 194 spanning the shank 186 and the flutes 184 of the extended file 180. The metallic file flutes 184 and the non-metallic file shank 186 also may be fixedly mechanically associated.

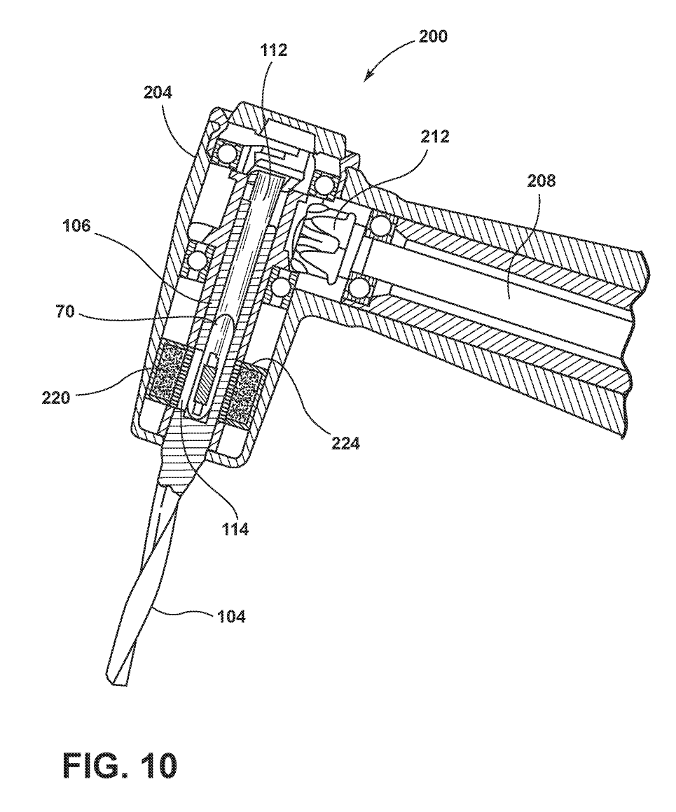

FIG. 10 shows a handpiece 200 with an augmented contra-angle cover 204 and an extended file 100. Any of the files 100, 120, 140, 160, 180 are contemplated for use with the handpiece 200. Augmentation of the contra-angle cover 204 facilitates accommodating the extended shank 106 of the extended file 100. The handpiece 200 includes a drive shaft 208 and a drive gear 212. The file 100 includes flutes 104 and an extended shank 106, disposed in the handpiece 200. The shank 106 defines an access bore 112 and an open window 114. The shank 106 may be similar in size to the shank of the file shown in FIG. 4 or FIG. 5. The shank 106 of the file 100 shown in FIG. 4 or FIG. 5 may be proportional to the file of FIG. 4 or FIG. 5, respectively, and similarly to the proportion of the shank of FIG. 10 to the file of FIG. 10. The transponder shown in FIG. 10 may be representative of the transponder 70 shown in FIG. 3A or the transponder 90 shown in FIG. 3B.

As shown in FIG. 10, the handpiece 200 features an RFID interrogator coil 220 surrounded on its outside surfaces by an electro-magnetic shielding 224. The tag antenna coil of the transponder 70 is shown disposed within, and exposed to, the RFID interrogator coil 220 through the open window 114. The augmented contra-angle cover 204 is configured to accommodate the RFID interrogator coil 220. The augmented contra-angle cover 204 is configured to accommodate the electro-magnetic shielding 224. Empty space depicted within the contra-angle cover 204 proximal and/or distal to the electro-magnetic shielding 224 may accommodate other components (not shown). The other components may be electronic and/or mechanical. Design of the augmented contra-angle cover 204 may minimize the space depicted.

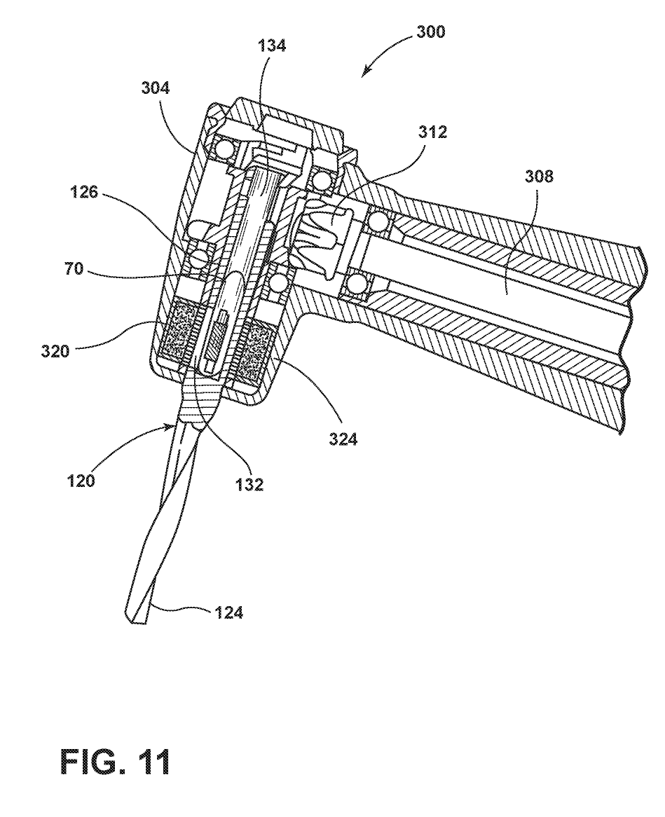

FIG. 11 shows a handpiece 300 with an augmented contra-angle cover 304 and an extended file 120. Augmentation of the contra-angle cover 304 facilitates accommodating the extended shank 126. The handpiece 300 includes a drive shaft 308 and drive gear 312. The file 120 includes flutes 124 and an extended shank 126, disposed in the handpiece 300. The shank 126 defines an access bore 134 and an open window 132. The shank 126 may be smaller in size than the shank of the file shown in FIG. 4, FIG. 5 or FIG. 10. The shank of the file shown in FIG. 4, FIG. 5 or FIG. 10 may be larger proportionally to the file of FIG. 4, FIG. 5 or FIG. 10, respectively, than the shank of FIG. 11 is to the file of FIG. 11. In length, the transponder 70 shown in FIG. 11 disposed within the shank 126 may be greater than, smaller than or similar to the transponder shown in FIG. 5 or FIG. 10. The transponder shown in FIG. 11 may be representative of the transponder 70 shown in FIG. 3A or the transponder shown in FIG. 3B.

As shown in FIG. 11, the handpiece 300 features an RFID interrogator coil 320 surrounded on its outside surfaces by an electro-magnetic shielding 324. The tag antenna coil of the transponder 70 is shown disposed within, and exposed to, the RFID interrogator coil 320 through the open window 132. The size and/or shape of the open window 132 relative to the transponder 70, may be similar to or different from the size and/or shape of the open window shown in FIG. 10 relative to the transponder of FIG. 10. As depicted in FIG. 11, the augmented contra-angle cover 304 is configured to accommodate the RFID interrogator coil 320. The augmented contra-angle cover 304 is also configured to accommodate the electro-magnetic shielding 324. The size and/or shape of the RFID interrogator coil 320 and/or of the electro-magnetic shielding 324 may be similar to or different from the size and/or shape of the RFID interrogator coil and/or of the electro-magnetic shielding, respectively, shown in FIG. 10. The empty space depicted in FIG. 11 within the contra-angle cover 304 proximal and/or distal to the electro-magnetic shielding 324 may accommodate other components (not shown). The other components may be electronic and/or mechanical. The empty space in FIG. 11 may be smaller than the empty space depicted in FIG. 10.

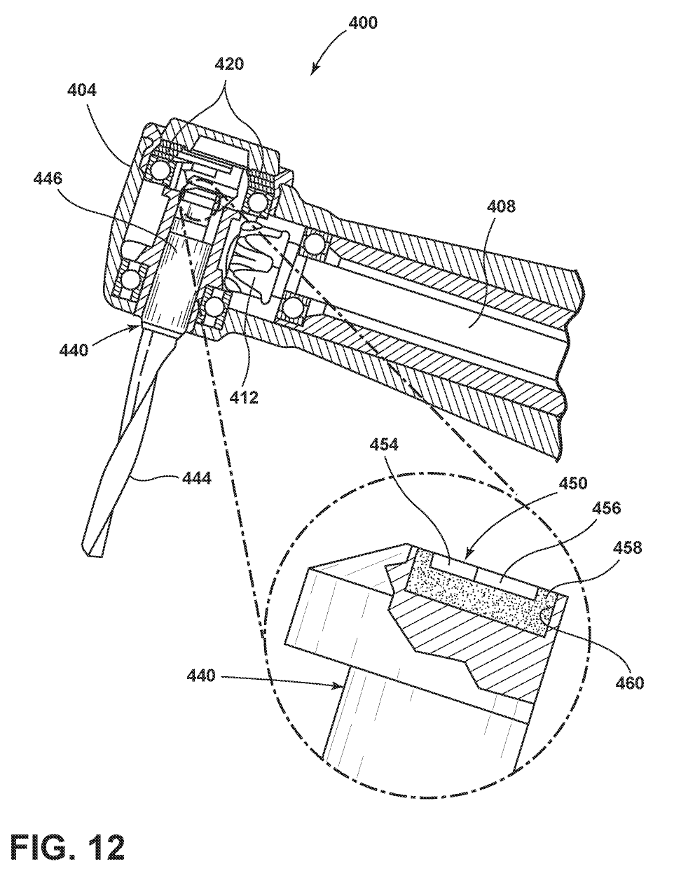

FIG. 12 shows a handpiece 400 with a contra-angle cover 404 and a file. The handpiece 400 includes a drive shaft 408 that powers a drive gear 412. The handpiece includes a RFID interrogator coil 420. In FIG. 12, the file 440 includes file flutes 444 and a file shank 446. The file 440 contains a transponder 450, as shown in inset of detail of the proximal end of the file. The transponder 450 may be a high-field communication device transponder. The transponder 450 includes a RFID chip 454 and a RFID antenna 456. A glue/polymer 458 secures the transponder 450 in an end recess 460 at the proximal end of the file 440. The transponder 450 may be representative of the transponder shown in FIG. 3B. The transponder 450 may be sufficiently small that extension of the shank is not necessary for the file to accommodate the transponder. The transponder 450 may be accommodated in the file 440 in a recess at the proximal end of the file. The transponder 450 may be affixed in the file 440, as by a glue and/or polymer that integrates the transponder with the file. The glue/polymer keeps the transponder 450 spaced from metal of the file, facilitating suitable development of an electromagnetic field for communication. The transponder 450 may communicate with the RFID interrogator coil 420, even with the transponder disposed at least partly outside of the RFID interrogator coil. The RFID interrogator coil 420 may be at least partly proximal to the transponder 450 or partly distal to the transponder. As shown, the transponder 450 may be entirely outside a field of the RFID interrogator coil 420. The RFID interrogator coil 420 may be sufficiently compact to be disposed within the contra-angle cover 404 with minimal or no augmentation of the contra-angle cover.

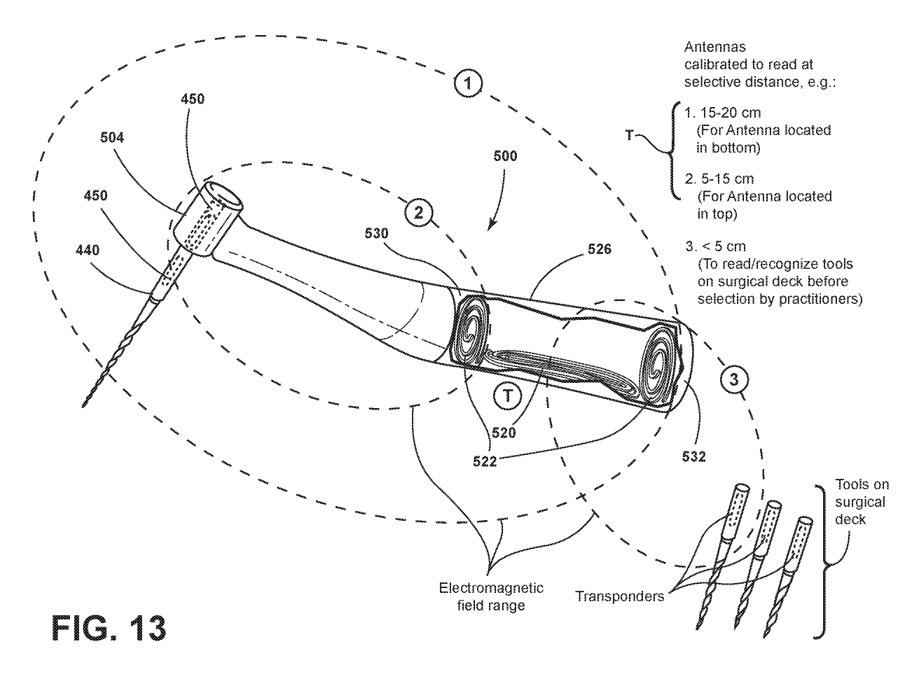

FIG. 13 shows several possible, non-exclusive, locations of transponders in or near a surgical handpiece 500. The transponders are shown associated with surgical tools 440 to which they may be affixed. The transponders 450 may be high-field communication device transponders including the types shown in FIG. 3A and/or FIG. 3B. FIG. 13 shows several possible, non-exclusive, locations of RFID interrogator coils 520 associated with the handpiece 500. The handpiece 500 is shown featuring a lower handpiece grip 526. The RFID interrogator coil 520 may be disposed at the top of the lower handpiece grip 526 and/or at the bottom of the lower handpiece grip. Also shown are some representative electromagnetic fields associated with communication ranges of one or more than one of the transponders with one or more than one of the RFID interrogator coils 520. The transponders 70, 90 450 and/or RFID interrogator coils 520 may be selectively configured to communicate over several of the ranges shown, allowing for robust identification and/or tracking of surgical tools, including files, for use with the handpiece 500.

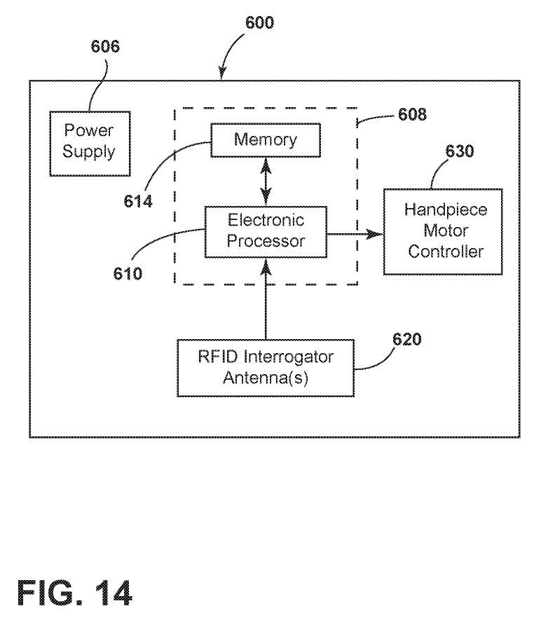

FIG. 14 shows a block diagram of a dental tool recognition system 600 for the handpiece 500 shown in FIG. 13 provided with a corresponding console. The dental tool recognition system 600 includes a power supply 606 to provide power to the various components including a data processing module 608 that includes an electronic processor 610 and a memory 614. The electronic processor 610 obtains tool information from tools having transponders via one or more RFID antennas 620. The diagram 600 includes a handpiece motor controller 630 that is controlled by the electronic processor 610.

In some embodiments, the electronic processor 610 is a microprocessor, an application-specific integrated circuit (“ASIC”), or other suitable electronic processing device having the memory 614. In some embodiments, the memory 614 is a non-transitory computer-readable medium including random access memory (“RAM”), read-only memory (“ROM”), or other suitable non-transitory computer-readable medium. Other types of memory 614 and circuitry are contemplated. The memory 614 of the data processing module 608 may store sets of operating system parameter values of the handpiece appropriate to one or more specific surgical tools, such as files. The data processing module 608 receives tool or file information from the tools or files attached to or disposed near the handpiece 500 as shown in FIG. 13.

In one embodiment, the power supply 606 is located in a console and the other components are disposed in the handpiece 500. Other arrangements are contemplated wherein the handpiece 500 is a wireless battery powered device.

In operation, the data processing module 608 is a second electronic module that receives information from a first electronic module that includes a transponder 70, 90, 450 or other RFID device secured on a tool.

Apparatuses and methods disclosed may involve some or all of the features of the illustrative apparatus and/or some or all of the steps of the illustrative methods. The steps of the methods may be performed in an order other than the order shown and described herein. Some embodiments may omit steps shown and described in connection with the illustrative methods. Some embodiments may include steps that are not shown and/or are not described in connection with the illustrative methods.

What is claimed is:

1. A tool recognition system comprising:
a tool selected for a surgical procedure, the tool defining a longitudinal axis and including:
a distal portion configured to perform at least part of the surgical procedure; and
a proximal portion, the proximal portion including a first electronic module, the first electronic module storing tool-identifying electronic information; and
a handpiece configured to receive the tool and including a second electronic module configured to communicate electronically with the first electronic module;
wherein, when the tool is disposed in the handpiece, the first electronic module electronically transmits the tool-identifying information to the second electronic module.

2. The tool recognition system of embodiment 1, wherein the first electronic module includes a coil configured as a radio-frequency identification (RFID) tag antenna with a coil axis substantially collinear with the longitudinal axis.

3. The tool recognition system of claim 1, wherein the handpiece is configured to control a function of the tool on a basis of the tool-identifying information transmitted to the second electronic module.

4. The tool recognition system of claim 3, wherein the handpiece is configured to control a function of the tool selected from the group consisting of rotary speed, torque, acceleration rate, deceleration rate, reciprocation linear stroke length, clockwise reciprocation, angular extend, counterclockwise reciprocation angular extent, reciprocation frequency, light emission wavelength, light emission intensity and mechanical vibration frequency.

5. The tool recognition system of claim 1, wherein the second electronic module includes a coil that is configured as a radio-frequency identification (RFID) interrogator antenna, and when the handpiece receives the tool, at least a portion of the first electronic module is disposed within the coil of the radio-frequency identification interrogator antenna.

6. The tool recognition system of embodiment 1, wherein the first electronic module further includes non-tool-identifying information.

7. The tool recognition system of claim 6, wherein the non-tool-identifying information includes at least one from a group consisting of a record of use of the tool and data corresponding to an environmental condition of the tool.

8. The tool recognition system of claim 6, wherein the handpiece is further configured to control at least one function of the tool on a basis of the tool-identifying information and at least another function of the tool on a basis of the non-tool-identifying information.

9. The tool recognition system of claim 6, wherein, when the tool is disposed in the handpiece, the non-tool- identifying information is electronically modified via a transmission from the second electronic module.

10. The tool recognition system of claim 1, wherein the tool-identifying information includes a value of an operating parameter of the handpiece corresponding to an operating function.

11. The tool recognition system of claim 10, further comprising a data processing module electronically storing sets of operating parameter values of the handpiece appropriate to specific surgical tools, and the data processing module configured to retrieve, on the basis of the information, the value appropriate to the identified tool.

12. An endodontic file recognition apparatus:
an endodontic file defining a longitudinal axis and including:
a fluted portion configured to perform at least part of a root canal procedure; and
a shank portion, the shank portion at least partly encasing a radio-frequency identification (RFID) tag; and
an endodontic drill configured to removably receive the shank portion and to control at least one function of the file during the procedure, the drill including an RFID interrogator antenna coil configured to communicate electronically with the RFID tag.

13. The endodontic file recognition apparatus of claim 12
wherein, when the shank portion is received in the drill, the interrogator coil is substantially collinear with the longitudinal axis and the RFID tag electronically transmits to the interrogator coil information identifying the file, the information determining at least one operating parameter of the drill corresponding to the at least one function.

14. An endodontic drill comprising:
a first section configured to removably receive a shank of an endodontic file, the file at least partly encasing a radio-frequency identification (RFID) tag, the first section further configured to move the file during a root canal procedure; and
a second section covering the first section and including an RFID interrogator antenna coil configured to accommodate the shank and to communicate electronically with the RFID tag.

15. The endodontic drill of claim 14
wherein, when the shank is received in the first section, the RFID tag electronically transmits information identifying the file to the interrogator coil, the information determining an operating parameter of the drill corresponding to the motion of the file.

16. The endodontic drill of claim 14, wherein, when the shank portion is coupled with the drill:
the tag coil is disposed at least partly within the interrogator coil;
the interrogator coil electronically activates the RFID tag; and
the interrogator coil electronically receives a version of the information from the RFID tag, the version determining a characteristic of the motion.

17. The endodontic drill of claim 15, wherein the characteristic of the motion for the file is selected from an electronic memory to correspond to the information received from the RFID tag.

18. A surgical tool for use in a surgical procedure, the tool defining a longitudinal axis and comprising:
a distal portion configured to perform at least part of the procedure; and
a proximal portion configured to releasably couple with a surgical handpiece, the proximal portion including a radio- frequency identification (RFID) tag and an RFID tag antenna, the tag storing electronic information characterizing the tool;
wherein, the handpiece is configured to control a motion of the tool during the procedure and includes an RFID interrogator antenna coil configured to:
communicate electronically with the RFID tag.

19. The tool of claim 18, wherein the tool is a dental tool selected from a group consisting of files, broaches, burrs, reamers and drill bits.

20. The tool of claim 18, wherein the characteristic of the motion is optimized for the tool as identified by the information received from the RFID tag.

21. The tool recognition system of claim 1, wherein a system parameter is electronically set that corresponds to a status of the tool or a confirmation of the status of the tool.

22. The tool recognition system of claim 21, wherein the status of the tool includes at least one from a group consisting of the identity of the tool, a measure of prior usage of the tool, a measure of current usage of the tool, a measure of readiness of the tool for use in the surgical procedure, location of the tool within the practitioner's facility, and a count of sterilization cycles to which the tool has been subjected.

23. The tool recognition system of claim 21, wherein the second electronic module is disposed in a distribution facility configured to store the tool prior to distribution to a user of the surgical procedure.

24. The tool recognition system of claim 21, wherein the second electronic module is disposed in a holder in a practitioner's facility and the holder is configured to hold the tool prior to use of the tool.
